(19) United States
(12) Patent Application Publication
(10) Pub. No.: US 2007/0099849 A1
(43) Pub. Date: May 3, 2007

(54) Co-Therapy for the Treatment of Migraine Comprising Anticonvulsant Derivatives and Anti-Migraine Agents

(76) Inventor: Ian R. Livingstone, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/608,276

(22) Filed: Dec. 8, 2006

Related U.S. Application Data
(63) Continuation of application No. 10/373,488, filed on Feb. 25, 2003.
(60) Provisional application No. 60/359,894, filed on Feb. 26, 2002.

(51) Int. Cl. A61K 31/70; A61K 31/255; A61K 31/36; A61K 31/35

(52) U.S. Cl. 514/23; 514/459; 514/464; 514/517

(57) ABSTRACT

The present invention describes a method for the treatment and/or prevention of migraine and associated symptoms (nausea, vomiting, photophobia, phonophobia, etc.) comprising co-therapy with a therapeutically effective amount of one or more anti-migraine agents and one or more anticonvulsant derivatives.

### CROSS REFERENCE TO RELATED APPLICATION

This application claims the benefit of U.S. Provisional Application 60/359,894, filed on Feb. 26, 2002, which is incorporated by reference herein in its entirety.

### BACKGROUND OF THE INVENTION

Migraine is a chronic, episodic and debilitating clinical condition that is diagnosed by the presence of moderate to severe pulsating unilateral headaches lasting between 4 and 72 h. Additionally, the headache is sometimes associated with temporary sensory (photophobia and phonophobia) and/or gastrointestinal (nausea, vomiting) disturbances. Migraine headaches can present without or with aura.

Migraine without aura is defined by at least five attacks fulfilling the following criteria: (a) the headache attacks lasting 4-72 hours with the headache having at least two of the following features: unilateral location, pulsating quality, moderate or severe intensity with a direct influence on activities of daily living, and aggravation by walking up stairs or similar routines; (b) during the headache at least one of the following occurs: nausea and/or vomiting, photophobia or phonophobia (Classification and diagnostic criteria for headache disorders, cranial neuralgias and facial pain. Headache Classification Committee of the International Headache Society. Cephalalgia 1988; 8 Suppl 7:1-96).

Migraine with aura is defined by at least two attacks accompanied by at least 3 of the 4 following features: (a) one or more fully reversible aura symptoms; (b) at least one aura symptom which develops gradually over more than four minutes or two or more symptoms which occur in succession; (c) no aura symptom that lasts more than 60 minutes; (d) the headache begins prior to, simultaneously with or following the aura, with a free interval between aura and headache of less than about 60 minutes (Classification and diagnostic criteria for headache disorders, cranial neuralgias and facial pain. Headache Classification Committee of the International Headache Society. Cephalalgia 1988; 8 Suppl 7:1-96).

The clinical profiles of patients with migraine headaches are represented by migraine without aura (about 70% of migraineurs) and migraine with aura (about 30%). Migraine without aura is also known as common migraine and typically has an average duration of about 18 to 24 hours. Pain is usually unilateral, but it can alternate sides or be bilateral during an attack. Migraine with aura can be associated with visual disturbances and the aura usually develops gradually over 5-20 min and usually lasts less than 60 minutes. Migraine with aura may be sequentially associated with attacks without aura. The most common form of migraine with aura is migraine with typical aura also known as classical migraine. Headache pain commences within 60 minutes of the end of the aura. Other less common types of migraine headaches exist and include, but are not limited to, migraine with prolonged aura which is associated with aura symptoms that last longer than 60 minutes; migraine aura without headache; migraine with acute onset aura; basilar migraine which can be associated with vertigo, gait disturbances and/or loss of consciousness; ophthalmoplegic migraine associated with ocular paralysis, diplopia and ptosis; retinal migraine; and familial hemiplegic migraine associated with hemiparesis or hemiplegia (Migraine. Cognos. Decision Resources, 2000).

Pharmacological interventions for the therapeutic management of migraine can be categorized into two general strategies: preventive approaches and treatments to relieve the pain and associated symptomatology or abortive therapy.

The objective of the preventive (prophylactic) therapy is to reduce the frequency of the migraine attacks, reduce the severity and/or shorten the duration of the attacks. Prophylactic treatments for migraine include anticonvulsants, antidepressants, beta blockers, calcium channel blockers nonsteroidal anti-inflammatory drugs (NSAIDs), and serotonin receptor antagonists. Many of these agents are used off-label in migraine prophylaxis. (Migraine. Cognos. Decision Resources, 2000).

Based on clinical studies, specific agents within the classes of antidepressants and beta-blockers have been shown to have the highest efficacy and the best adverse side effects profile.

Anticonvulsants used in migraine prophylaxis include, but are not limited to, topiramate (Ortho-McNeil's TOPAMAX), valproic acid (Abbott's DEPAKENE), divalproex sodium (Abbott's DEPAKOTE), and gabapentin (Warner-Lambert's NEURONTIN).

Antidepressants used in migraine prophylaxis include, but are not limited to, tricyclic antidepressants such as amitriptyline (Schering's ETRAFON, ICN's LIMBITROL, Banyu's TRYPTANOL, Bayer's SAROTEN, Roche's LAROXYL, Astra Zeneca's ELAVIL, and generics), nortriptyline (Novartis' PAMELOR, and generics), clomipramine (Novartis' ANAFRANIL, and generics), imipramine (Novartis' TOFRANIL, and generics), doxepin (Pfizer's SINEQUAN, and generics); mono-amine oxidase inhibitors such as phenelzine (Parke-Davis' NARDIL); selective serotonin reuptake inhibitors such as fluoxetine (Eli Lilly's PROZAC, SARAFEM and generics), fluvoxamine (Solvay's LUVOX), citalopram (Lundbeck's CIPRAMIL, and Forest's CELEXA); and selective serotonin noradrenaline reuptake inhibitors such as venlafaxine (Wyeth-Ayerst's EFFEXOR XR).

Beta blockers used in migraine prophylaxis include, but are not limited to, metoprolol (Astra-Zeneca's TOPROL-XR, Novartis' LOPRESSOR, and generics), atenolol (Astra Zeneca's TENORMIN, TEMORETIC, and generics), propanolol (Wyeth-Ayerst's INDERAL, and generics), timolol (Merck, Sharp and Dohme's BLOCADREN, Falcon's TIMOLOL, and generics), and nadolol (Bristol-Myers Squibb's Monarch's CORGARD/SOLGOL, Dainippon's NADIC, and generics).

Calcium channel blockers used in migraine prophylaxis include, but are not limited to, verapamil (Knoll's ISOPTIN, Schwarz's Verelan, Searle's Covera and CALAN, and generics), lomerizine (TERRANAS from Nippon Organon's), flunarizine (SIBELIUM from Janssen Pharmaceutica), diltiazem (Biovail CARDIZEM, and generics), nimodipine (Bayer, NIMOTOP and ESTEVE), zucapsaicin (Civamide from Winston Laboratories), and dotarizine (from Mylan/Ferrer).

Nonsteroidal anti-inflammatory drugs used in migraine prophylaxis include, but are not limited to, naproxen (Roche Laboratories' Naprosyn and generics) and ketoprofen (Wyeth-Ayerst's ORUDIS and ORUVAIL and generics).

Serotonin receptor antagonists used in migraine prophylaxis include, but are not limited to, Pizotifen (Novartis's SANOMIGRAN/PIZOTYLINE), methysergide (Novartis' SANSERT/DESERIL, and generics), and cyproheptadine (Merck's PERIACTIN).

Abortive treatments in the management of migraine headache (the relief of the pain and/or associated symptomology of migraine attacks) include analgesics and combinations, antiemetics, ergot derivatives, nonsteroidal anti-inflammatory drugs, and triptans. Neuropeptide antagonists are also been studied. (Migraine. Cognos. Decision Resources, 2000).

Analgesics and combinations (including combinations with other drugs such as antiemetics) for the abortive treatment of migraine include, but are not limited to aspirin, acetaminophen, paracetamol, meperidine, codeine, hydrocodone, Novartis' FIORICET or Forests' ESGIC or generics (combination of acetaminophen and butalbital and caffeine), FIORINAL or generics (combination of aspirin, butalbital and caffeine, Novartis), MIGPRIV or generics (combination of aspirin and metoclopromide; Sanofi-Synthelabo), MIDRIN/MIDRID or generics (combination of acetaminophen and dichloralphenazone; Carnick), Sanofi-Synthelabo's PARAMAX or Dolorgiet's MIGRAENERTON or generics (combination of paracetamol and metoclopramide), Abbott's VICODIN or generics (combination of acetaminophen and hydrocodone), STADOL NS (butorphanol nasal spray; Bristol-Myers Squibb), Boehringer Ingelheim's LONARID or Pfizer's MIGRALEVE or generics (combination of paracetamol and codeine).

Antiemetics for the abortive treatment of migraine include, but are not limited to, metoclopramide (SmithKline Beecham's MAXOLON, Robin's REGLAN, and generics), domperidone (Janssen Pharmaceutica's MOTILIUM, and generics), prochlorperazine (SmithKline Beecham's COMPAZINE, and generics), and promethazine (Wyeth-Ayerst's PHENERGAN/MEPERGAN, and generics).

Ergot derivatives for the abortive treatment of migraine include, but are not limited to, dihydroergotamine (Novartis DHE-45, MIGRANAL nasal spray), ergotamine (Lotus Biochemical's ERGOMAR, and generics), and combination of ergotamine with caffeine (Novartis' CAFERGOT, Organon's WIGRAINE, and generics).

Nonsteroidal anti-inflammatory drugs for the abortive treatment of migraine include, but are not limited to, aspirin, ibuprofen, diclofenac (Novartis' VOLTAREN, and generics), naproxen (Roche's NAPROSYN, and generics) and ketoprofen (Wyeth-Ayerst's ORUDIS and ORUVAIL, and generics).

Triptans for the abortive treatment of migraine include, but are not limited to, sumatriptan (IMITREX/IMIGRAN, Glaxo Wellcome), naratriptan (AMERGE from Glaxo Wellcome), rizatriptan (MAXALT from Merck), zolmitriptan (ZOMIG from Astra Zeneca), eletriptan (RELPAX from Pfizer), frovatriptan (MIGUARD from Vernalis/Elan/Menarini), and almotriptan (AXERT from Pharmacia).

Neuropeptide antagonists which may be useful in prophylactic as well as abortive therapy of migraine include, but are not limited to, the following agents: calcitonin gene-related peptide antagonist (BIBN 4096 from Boehringer Ingelheim), and substance P antagonists such as dapitant (Aventis's ERISPANT), lanepitant (Lilly's LY-303870) and FK-888 from Fujisawa.

Drugs for prophylactic treatment of migraine must be taken daily and many are associated with undesired adverse effects. For example, the use of methysergide carries with it the danger of retroperitoneal fibrosis. For nonsteroidal anti-inflammatory drugs the need for high dosages for effectiveness is a drawback. Tricyclic antidepressants are associated with multiple side effects including sedation, weight gain and anticholinergic effects including dry mouth, blurred vision, constipation, cognitive impairment, and urinary retention. Monoamine oxidase inhibitors are often associated with side effects which include orthostatic hypotension, hypertensive crisis, body weight gain, insomnia and sexual dysfunction. Selective serotonin reuptake inhibitors side effects include nausea, diarrhea, constipation, sleep impairment, sexual dysfunction, and anxiety and the risk for serotonin syndrome. Venlafaxine can be associated with unwanted cardiovascular effects, sedation, anticholinergic effects, gastrointestinal disturbances, and sexual dysfunction. Valproic acid side effects include drowsiness, nausea, fatigue, tremor, and weight gain. In many cases it is the side effects that are the cause for noncompliance and self-discontinuation. In addition, it has been estimated that the probability of success with any one of the available prophylactic anti-migraine drugs is about 60-70% (Harrison's Principles of Internal Medicine, eds. Isselbacher et al., McGraw-Hill, Inc., New York, 1994, p/69).

Compounds of Formula (I):

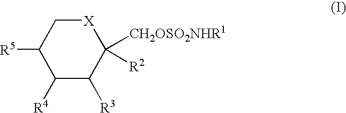

are structurally novel antiepileptic compounds that are highly effective anticonvulsants in animal tests (MARYANOFF, B. E, NORTEY, S. O., GARDOCKI, J. F., SHANK, R. P. AND DODGSON, S. P. J. Med. Chem. 1987, 30, 880-887; MARYANOFF, B. E., COSTANZO, M. J., SHANK, R. P., SCHUPSKY, J. J., ORTEGON, M. E., AND VAUGHT J. L. Bioorg. Med. Chem. Lett. 1993, 3, 2653-2656; SHANK, R. P., GARDOCKI, J. F., VAUGHT, J. L., DAVIS, C. B., SCHUPSKY, J. J., RAFFA, R. B., DODGSON, S. J., NORTEY, S. O., MARYANOFF, B. E. Epilepsia 1994, 35, 450-460; MARYANOFF B E, COSTANZO M J, NORTEY S O, GRECO M N, SHANK R P, SCHUPSKY J J, ORTEGON M P, VAUGHT J L. J. Med. Chem. 1998, 41, 1315-1343). These compounds are covered by three U.S. patents: U.S. Pat. No. 4,513,006, No. 5,242,942, and No. 5,384,327. One of these compounds 2,3:4,5-bis-O-(1-methylethylidene)-β-D-fructopyranose sulfamate known as topiramate has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world.

Compounds of Formula (I) were initially found to possess anticonvulsant activity in the traditional maximal electroshock seizure (MES) test in mice (SHANK, R. P., GARDOCKI, J. F., VAUGHT, J. L., DAVIS, C. B., SCHUPSKY, J. J., RAFFA, R. B., DODGSON, S. J., NORTEY, S. O., and MARYANOFF, B. E., Epilepsia 1994, 35, 450-460). Subsequent studies revealed that Compounds of Formula (I) were also highly effective in the MES test in rats. Topiramate was also found to effectively block seizures in several rodent models of epilepsy (J. NAKAMURA, S. TAMURA, T. KANDA, A. ISHII, K. ISHIHARA, T. SERIKAWA, J. YAMADA, and M. SASA, Eur. J. Pharmacol. 1994, 254, 83-89), and in an animal model of kindled epilepsy (A. WAUQUIER and S. ZHOU, Epilepsy Res. 1996, 24, 73-77).

Ehrenberg et al., in U.S. Pat. No. 5,999,380 disclose the use of compounds of formula (I) to treat migraines in non-epileptic patients. More particularly, Ehrenberg et al., disclose the use of compounds of formula (I) for reducing the frequency or severity of migrainous episodes in non-epileptic patients.

It has unexpectedly been found that co-therapy comprising one or more anticonvulsant derivatives, compounds of formula (I), and one or more drugs used for the prevention and/or treatment of migraine is useful for the treatment and/or prevention of migraine.

### SUMMARY OF THE INVENTION

The present invention is directed to the treatment and/or prevention of migraine with co-therapy comprising administration of a therapeutically effective amount of one or more anti-migraine agents and one or more compounds of formula (I)

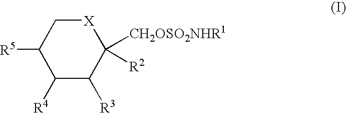

wherein

X is CH₂ or oxygen;

R¹ is hydrogen or alkyl; and

R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II):

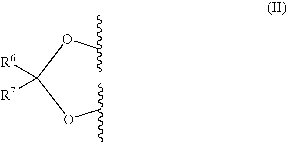

wherein

R⁶ and R⁷ are the same or different and are hydrogen, lower alkyl or are alkyl and are joined to form a cyclopentyl or cyclohexyl ring.

The present invention is further directed to a method for treating nausea, vomiting, photophobia and/or phonophobia, preferably nausea, photophobia and/or phonophobia, associated with migraine headaches in a subject in need thereof comprising co-therapy with a therapeutically effective amount of a compound of formula (I) and an anti-migraine agent. Preferably, the compound of formula (I) is topiramate and the anti-migraine agent is an abortive agent. More preferably the compound of formula (I) is topiramate and the anti-migraine agent is a triptan.

In an embodiment of the present invention, the compound of formula (I) is topiramate. In an embodiment of the present invention, the anti-migraine agent is a prophylactic agent. In another embodiment of the present invention, the anti-migraine agent is an abortive agent.

In an embodiment of the present invention, the anti-migraine agent is a triptan. Preferably, the triptan is selected from the group consisting of sumatriptan (IMITREX/IMIGRAN, Glaxo Wellcome), naratriptan (AMERGE from Glaxo Wellcome), rizatriptan (MAXALT from Merck), zolmitriptan (ZOMIG from Astra Zeneca), eletriptan (RELPAX from Pfizer), frovatriptan (MIGUARD from Vernalis/Elan/Menarini), and almotriptan (AXERT from Pharmacia).

In an embodiment of the present invention is a method for the treatment and/or prevention of migraine which comprises co-therapy with a therapeutically effective amount of topiramate and an anti-migraine agent, wherein the anti-migraine agent is a prophylactic agent. In another embodiment of the present invention is a method for the treatment and/or prevention of migraine which comprises co-therapy with a therapeutically effective amount of topiramate and an anti-migraine agent, wherein the anti-migraine agent is a an abortive agent.

In an embodiment of the present invention is a method for the treatment and/or prevention of migraine which comprises co-therapy with a therapeutically effective amount of topiramate and a compound selected from the group consisting of analgesics, antiemetics, ergot derivatives, nonsteroidal anti-inflammatory drugs, triptans, neuropeptide antagonist, anticonvulsants, antidepressants, beta-blockers, calcium channel blockers and serotonin receptor antagonists.

In an embodiment of the present invention is a method for the treatment of migraine which comprises co-therapy with a therapeutically effective amount of topiramate and a compound selected from the group consisting of analgesics, antiemetics, ergot derivatives, nonsteroidal anti-inflammatory drugs, triptans and neuropeptide antagonists.

In an embodiment of the present invention is a method for the prevention of migraine which comprises co-therapy with a therapeutically effective amount of topiramate and a compound selected from the group consisting of anticonvulsants, antidepressants, beta-blockers, calcium channel blockers, nonsteroidal anti-inflammatory drugs and serotonin receptor antagonists.

In an embodiment of the present invention is a method for the treatment and/or prevention of migraine which comprises co-therapy with a therapeutically effective amount of topiramate and a compound selected from the group consisting of antidepressants, beta blockers and triptans.

### DETAILED DESCRIPTION OF THE INVENTION

As used herein, the term “migraine” shall mean a chronic, episodic and debilitating clinical condition that is diagnosed by the presence of moderate to severe pulsating unilateral headaches lasting between 4 and 72 h, which includes migraine without aura and migraine with aura.

As used herein, “migraine without aura” shall mean at least five attacks fulfilling the following criteria: (a) the headache attack lasts 4-72 hours with the headache having at least two of the following features: unilateral location, pulsating quality, moderate or severe intensity with direct influence on activities of daily living, and aggravation by walking up stairs or similar routines; and (b) during the headache at least one of the following occurs: nausea and/or vomiting, and photophobia and phonophobia.

As used herein, “migraine with aura” shall mean at least two attacks accompanied by at least 3 of the 4 following features: (a) one or more fully reversible aura symptoms; (b) at least one aura symptom which develops gradually over more than four minutes or two or more symptoms which occur in succession; (c) no aura symptom which lasts more than 60 minutes; (d) a headache occurs prior to, simultaneously with or following the aura, with a free interval between aura and headache of less than about 60 minutes.

As used herein, the term “prevention” shall include the prevention of migraine attacks, a decrease in the frequency of migraine attacks, a decrease in the severity of migraine attacks and/or a decrease in the duration of migraine attacks.

As used herein, the term “prophylactic agent” shall mean any pharmaceutical agent which may be used for the prevention or prophylaxis of migraine. Suitable examples include, but are not limited to pharmaceutical agents in the classes of anticonvulsants, antidepressants, beta blockers, calcium channel blockers, nonsteroidal anti-inflammatory drugs (NSAIDs) and serotonin receptor antagonist.

As used herein. the term “abortive agent” shall mean any pharmaceutical agent which may be used for the treatment of migraine. Suitable examples include, but are not limited to pharmaceutical agents in the classes of analgesics and combinations, antiemetics, ergot derivatives, nonsteroidal anti-inflammatory drigs (NSAIDs), triptans and neuropeptide antagonists.

As used herein, the term “subject” refers to an animal, preferably a mammal, most preferably a human, who is the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes prevention and/or alleviation of the symptoms of the disease or disorder being treated. Wherein the present invention is directed to co-therapy comprising administration of one or more compound(s) of formula (I) and one or more anti-migraine agent(s), “therapeutically effective amount” shall mean that amount of the combination of agents taken together so that the combined effect elicits the desired biological or medicinal response. For example, the therapeutically effective amount of co-therapy comprising administration of a compound of formula (I) and an anti-migraine agent would be the amount of the compound of formula (I) and the amount of the anti-migraine agent that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the anti-migraine agent individually may or may not be therapeutically effective.

As used herein, the term “co-therapy” shall mean treatment of a subject in need thereof by administering one or more compounds of formula (I) with one or more anti-migraine agents, wherein the compound(s) of formula (I) and the anti-migraine agent(s) are administered by any suitable means, simultaneously, sequentially, separately or in a single pharmaceutical formulation. Where the compound(s) of formula (I) and the anti-migraine agent(s) are administered in separate dosage forms, the number of dosages administered per day for each compound may be the same or different. The compound(s) of formula (I) and the anti-migraine agent(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) and the anti-migraine agent(s) may be administered according to simultaneous or alternating regimens, at the same or different times during the course of the therapy, concurrently in divided or single forms.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

The anticonvulsant derivatives of the invention are of the following formula (I):

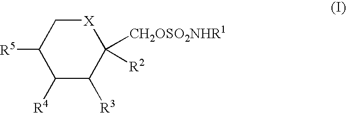

wherein

X is CH₂ or oxygen;

R¹ is hydrogen or alkyl; and

R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (II):

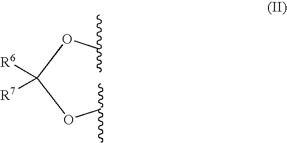

wherein

R⁶ and R⁷ are the same or different and are hydrogen, lower alkyl or are alkyl and are joined to form a cyclopentyl or cyclohexyl ring.

R¹ in particular is hydrogen or alkyl of about 1 to 4 carbons, such as methyl, ethyl and iso-propyl. Alkyl throughout this specification includes straight and branched chain alkyl. Alkyl groups for R², R³, R⁴, R⁵, R⁶ and R⁷ are of about 1 to 3 carbons and include methyl, ethyl, iso-propyl and n-propyl. When X is CH₂, R⁴ and R⁵ may combine to form a benzene ring fused to the 6-membered X-containing ring, i.e., R⁴ and R⁵ are defined by the alkatrienyl group ═C—CH═CH—CH═.

A particular group of compounds of formula (I) is that wherein X is oxygen and both R² and R³ and R⁴ and R⁵ together are methylenedioxy groups of the formula (II), wherein R⁶ and R⁷ are both hydrogen both alkyl or combine to form a spiro cyclopentyl or cyclohexyl ring, in particular where R⁶ and R⁷ are both alkyl such as methyl. A second group of compounds is that wherein X is CH₂ and R⁴ and R⁵ are joined to form a benzene ring. A third group of compounds of formula (I) is that wherein both R² and R³ are hydrogen.

The compounds of formula (I) may be synthesized by the following methods:

(a) Reaction of an alcohol of the formula RCH₂OH with a chlorosulfamate of the formula CISO₂NH₂ or CISO₂NHR¹ in the presence of a base such as potassium t-butoxide or sodium hydride at a temperature of about −20°to 25° C. and in a solvent such as toluene, THF, or dimethylformamide wherein R is a moiety of the following formula (III):

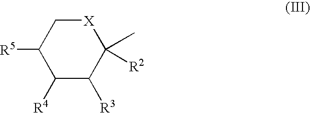

(b) Reaction of an alcohol of the formula RCH₂OH with sulfurylchloride of the formula SO₂Cl₂ in the presence of a base such as triethylamine or pyridine at a temperature of about −40°to 25° C. in a solvent such as diethyl ether or methylene chloride to produce a chlorosulfate of the formula RCH₂OSO₂Cl.

The chlorosulfate of the formula RCH₂OSO₂Cl may then be reacted with an amine of the formula R¹NH² at a temperature of abut 40 to 25° C. in a solvent such as methylene chloride or acetonitrile to produce a compound of formula (I). The reaction conditions for (b) are also described by T. Tsuchiya et al. in Tetrahedron Lett., 1978, 3365.

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by M. Hedayatullah in Tetrahedron Lett. 1975, 2455. The azidosulfate is then reduced to a compound of formula (I) wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula (II) may be obtained by the method of R. F. Brady in Carbohydr. Res. 1970, 14, 35 or by reaction of the trimethylsilyl enol ether of a R⁶COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by G. L. Larson et al. in J. Org. Chem. 1973, 38, 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such a diglyme, THF or toluene at a temperature of about 0° to 100° C., e.g. as described by H.O. House in “Modern Synthetic Reactions”, 2nd Ed., pages 45 to 144 (1972).

The compounds of formula (I) may also be made by the process disclosed US patents: U.S. Pat. No. 4,513,006, No. 5,242,942, and No. 5,384,327, which are incorporated by reference herein.

The compounds of formula (I) include the various individual isomers as well as the racemates thereof, e.g., the various alpha and beta attachments, i.e., below and above the plane of the drawing, of R², R³, R⁴ and R⁵ on the 6-membered ring. Preferably, the oxygen of the methylenedioxy group of formula (II) are attached on the same side of the 6-membered ring.

As used herein, the term “anti-migraine agent” shall include any pharmacological agent which may be used to treat or prevent migraine attacks (i.e. any pharmacological agent which may be used for the treatment or prophylaxis of migraine). Suitable examples include, but are not limited to, pharmacological agents in the classes of anticonvulsants, antidepressants, beta-blockers, calcium channel blockers, nonsteroidal anti-inflammatory agents, serotonin receptor antagonists, serotonin reuptake inhibitors, serotonin noradrenaline reuptake inhibitors, analgesics, antiemetics, ergot derivatives, triptans, neuropeptide antagonists and riboflavin (vitamin B2).

As used herein anticonvulsants includes, but are not limited to, valproic acid (usual daily oral dosage of 10 to 60 mg) (Abbott's DEPAKENE), divalproex sodium (usual daily oral dosage of 10 to 60 mg) (Abbott's DEPAKOTE), and gabapentin (usual daily oral dosage of 300 to 1800 mg for adults, with lower dosage levels for children) (Warner-Lambert's NEURONTIN).

As used herein antidepressants, include but are not limited, to tricyclic antidepressants such as amitriptyline (usual daily oral therapeutic dose range of 150-300 mg) (Schering's ETRAFON, ICN's LIMBITROL, Banyu's TRYPTANOL, Bayer's SAROTEN, Roche's LAROXYL, Astra Zeneca's ELAVIL, and generics), nortriptyline (usual daily oral therapeutic dose range of 50-150 mg) (Novartis' PAMELOR, and generics), clomipramine (usual daily oral therapeutic dose range of 100-250 mg) (Novartis' ANAFRANIL, and generics), imipramine (usual daily oral therapeutic dose range of 150-300 mg) (Novartis' TOFRANIL, and generics), doxepin (usual daily oral therapeutic dose range of 150-300 mg) (Pfizer's SINEQUAN, and generics); mono-amine oxidase inhibitors such as phenelzine (usual daily oral therapeutic dose range of 45-90 mg) (Parke-Davis' NARDIL); selective serotonin reuptake inhibitors such as fluoxetine (usual daily oral therapeutic dose range of 20-60 mg) (Eli Lilly's PROZAC, SARAFEM and generics), fluvoxamine (usual daily oral therapeutic dose range of 100-300 mg) (Solvay's LUVOX), citalopram (usual daily oral therapeutic dose range of 20-40 mg) (Lundbeck's CIPRAMIL, and Forest's CELEXA); and selective serotonin noradrenaline reuptake inhibitors such as venlafaxine (usual daily oral therapeutic dose range of 125-375 mg) (Wyeth-Ayerst's EFFEXOR).

Beta blockers include, but are not limited to, metoprolol (usual daily oral therapeutic dose of about 200 mg) (Astra-Zeneca's TOPOL-XL, Novartis' LOPRESSOR, and generics), atenolol (usual daily oral therapeutic dose of about 100 mg) (Astra Zeneca's TENORMIN and TEMORETIC, and generics), propanolol (usual daily oral therapeutic dose of about 160 mg) (Wyeth-Ayerst's INDERAL, and generics), timolol (usual daily oral therapeutic dose of about 20 mg) (Merck, Sharp and Dohme's BLOCADREN, Falcon's TIMOLOL, and generics), and nadolol (usual daily oral therapeutic dose of about 160 mg) (Bristol-Myers Squibb's-Monarch's CORGARD/SOLGOL, Dainippon's NADIC, and generics).

Calcium channel blockers include, but are not limited to, verapamil (usual daily oral dosage of 120 to 480 mg) (Knoll's ISOPTIN, Schwarz's Verelan, Searle's Covera and CALAN, and generics), lomerizine (TERRANAS from Nippon Organon's), flunarizine (SIBELIUM from Janssen Pharmaceutica), diltiazem (usual daily oral dosage of 120 to 360 mg) (Biovail CARDIZEM, and generics), nimodipine (usual daily oral dosage of 60 to 240 mg) (Bayer, NIMOTOP and ESTEVE), zucapsaicin (Civamide from Winston Laboratories), and dotarizine (from Mylan/Ferrer).

Nonsteroidal anti-inflammatory drugs include, but are not limited to, aspirin, ibuprofen, diclofenac (usual daily oral dosage of 50 to 200 mg) (Novartis' VOLTAREN, and generics), naproxen (usual daily oral dosage of 500 to 1000 mg) (Roche's NAPROSYN, and generics) and ketoprofen (usual daily oral dosage of 150 to 300 mg) (Wyeth-Ayerst's ORUDIS and ORUVAIL, and generics).

As used herein, serotonin receptor antagonists include, but are not limited to, pizotifen (Novartis's SANOMIGRAN/PIZOTYLINE), methysergide (Novartis' SANSERT/DESERIL, and generics), and cyproheptadine (usual daily oral dosage of 4 to 20 mg) (Merck's PERIACTIN).

Analgesics and combinations (including combinations with other drugs such as antiemetics) include, but are not limited to aspirin, acetaminophen, paracetamol, meperidine, codeine, hydrocodone, Novartis' FIORICET or Forests' ESGIC or generics (combination of acetaminophen and butalbital and caffeine), FIORINAL or generics (combination of aspirin, butalbital and caffeine, Novartis), MIGPRIV or generics (combination of aspirin and metoclopromide; Sanofi-Synthelabo), MIDRIN/MIDRID or generics (combination of acetaminophen and dichloralphenazone; Carnick), Sanofi-Synthelabo's PARAMAX or Dolorgiet's MIGRAENERTON or generics (combination of paracetamol and metoclopramide), Abbott's VICODIN or generics (combination of acetaminophen and hydrocodone), STADOL NS (butorphanol nasal spray; Bristol-Myers Squibb), Boehringer Ingelheim's LONARID or Pfizer's MIGRALEVE or generics (combination of paracetamol and codeine).

As used herein, antiemetics include, but are not limited to, metoclopramide (usual oral dosage of 10 to 15 mg q.i.d.) (SmithKline Beecham's MAXOLON, Robin's REGLAN, and generics), domperidone (Janssen Pharmaceutica's MOTILIUM, and generics), prochlorperazine (usual oral dosage of 5 to 20 mg q.i.d.) (SmithKline Beecham's COMPAZINE, and generics) and promethazine (usual oral dosage of 12.5 to 50 mg) (Wyeth-Ayerst's PHENERGAN/MEPERGAN, and generics).

Ergot derivatives include, but are not limited to, dihydroergotamine (Novartis DHE-45, MIGRANAL nasal spray), ergotamine (Lotus Biochemical's ERGOMAR, and generics), and combination of ergotamine with caffeine (Novartis' CAFERGOT, Organon's WIGRAINE, and generics).

Triptans that include, but are not limited to, sumatriptan (usual therapeutic oral dose of about 50 mg) (IMITREX/IMIGRAN, Glaxo Wellcome), naratriptan (usual therapeutic oral dose of about 2.5 mg) (AMERGE, Glaxo Wellcome), rizatriptan (usual therapeutic oral dose of 5-10 mg) (MAXALT, Merck), zolmitriptan (usual therapeutic oral dose of about 2.5 mg) (ZOMIG, Astra Zeneca), and newer triptans including but not limited to eletriptan (RELPAX, Pfizer), frovatriptan (MIGUARD, Vernalis/Elan/Menarini), and almotriptan (AXERT from Pharmacia).

As used herein, neuropeptide antagonists include but are not limited to the following agents: calcitonin gene-related peptide antagonist (BIBN 4096 from Boehringer Ingelheim), and substance P antagonists such as dapitant (Aventis's ERISPANT), lanepitant (Lilly's LY-303870) and FK-888 from Fujisawa.

Therapeutically effective dosage levels and dosage regimens for anticonvulsants, antidepressants, beta-blockers, calcium channel blockers, nonsteroidal anti-inflammatory drugs, serotonin receptor antagonists, analgesics, antiemetics, ergot derivatives, triptans, neuropeptide antagonists, and other pharmaceutical agents disclosed herein, may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http://www.pdrel.com) and other sources.

The effectiveness of co-therapy comprising administration of a therapeutically effective amount of one or more anti-migraine agents with one or more compounds of formula (I) to treat or prevent migraine is based on case studies and results from clinical trials, as described in more detail herein.

### Case Study 1

The patient was a female, age 15, with persistent daily headache with migraine features. Standard neurological workup including MRI scan was normal. The patient failed to respond to PERIACTIN (cyproheptadine HCl), nortriptyline and INDERAL (propranolol HCl). Her severe headaches did however respond to naratriptan. The patient was started on topiramate at 25 mg/daily, increasing to 75 mg/day with a significant improvement and resolution of the daily headaches; and a decrease in migraine headache frequency to approximately one per week. The improvement was noted with treatment including topiramate at a dosage level of 75 mg/day and INDERAL at 20 mg/day.

### Case Study 2

The patient was a male, age 41, with a lifelong history of refractory migraine (migraine without aura), averaging 8 migraines per month. The patient showed no response to CORGARD (nadolol) in combination with PROZAC (fluoxetine HCl) or CELEXA (citalopram HCl) or trazodone. Supplementation with riboflavin (vitamin B2) at 400 mg/day also resulted in no improvement. The patient was started on topiramate at 25 mg/day, with dosage increasing to 75 mg/day. Simultaneously, the beta-blocker, CORGARD dosage was decreased to 20 mg/day, with CELEXA at 20 mg/day also continued. At 75 mg/day topiramate, 20 mg/day CORGARD and 20 mg/day CELEXA, the patient reported a significant decrease in headache frequency, with no headache for up to four weeks.

### Case Study 3

The patient was a female, age 51, with a twenty-year history of severe refractory migraine with and without aura. The patient had modest symptomatic response to DEPAKOTE (valproic acid), which was discontinued due to weight gain. Only modest response was reported with INDERAL at 120 mg/day in combination with tricyclic antidepressants; while symptomatic response was reported to repeated frequent use of sumatriptan. The patient was started on topiramate at 50 mg/day increasing to 100 mg in the morning and 100 mg in the evening, in combination with INDERAL at 160 mg/day. The patient reported initial positive response, but the headaches broke through. After about 6 months, the patient was prescribed EFFEXOR XR (vanlafaxine HCl) at 37 mg/day, an increased topiramate dose of 125 mg in the morning and 150 mg in the evening, and 160 mg/day INDERAL. Modest but sustained improvement was reported with this combination.

### Clinical Protocol Study Trials #1 and #2

### Double-Blind, Placebo-Controlled, Parallel Group. Dose-Response Study

The primary objective of these studies was to evaluate the safety and efficacy of three doses of topiramate (50,100, and 200 mg/day) versus placebo in the prophylaxis of migraine based on the change in the monthly (28 day) migraine period rate from the Prospective Baseline Period to the Double-Blind Phase. The secondary objectives for the studies were to assess dose response relationship, and to evaluate the effect of prophylactic treatment with topiramate (50,100, and 200 mg/day) versus placebo on Health-Related Quality of Life (HRQL).

The studies were randomized, double-blind, placebo-controlled, parallel-group, multicenter studies. Male and female subjects were randomized equally to the four treatment groups. Subjects must have had an established history consistent with the diagnosis of migraine for at least six months, with or without aura, based on International Headache Society (IHS) criteria.

While the IHS criteria was used to establish the diagnosis at study entry, the evaluation of efficacy was based on migraine periods. A migraine period was defined as the length of time between the onset and cessation of painful migraine symptoms. This period could last up to, but no longer than, 24 hours. If the painful symptoms persisted more than 24 hours after their initial onset, this was considered to be a new, distinct migraine period. If symptoms recurred within 24 hours of the initial onset, this was considered part of the same, initial period. When an aura occurred, but successful abortive treatment prevented the headache from starting, this clinical situation was counted as a migraine period.

There were five phases in these studies: Baseline, Double-Blind, Blinded Transition, Open-Label Extension, and Taper/Exit, which are described in more detail below.

Baseline Phase:

The Baseline Phase lasted up to 42 days (including a maximum 14 day washout period) and included two periods: Washout and Prospective Baseline.

At Baseline Visit 1 (screening), subjects were evaluated to ensure that they met inclusion/exclusion criteria. In addition, a three month retrospective headache history was recorded. During the three months prior to Visit 1, subjects should have had an average of no more than 8 migraine attacks and no more than 15 total headache days (migraine plus non-migraine) per month. Eligible subjects then underwent other study procedures and were given a headache/medication record. Subjects completed headache records from Visit 1 onward throughout their study participation, documenting the occurrence of any headaches or auras, as well as the duration, severity, and symptomatology of any headache attacks. Subjects also recorded the use of any abortive/rescue medication taken for the relief of migraine or headache pain and associated symptoms, or during an aura to prevent migraine pain or relieve symptoms. In addition, for each migraine attack, subjects answered the questions on the headache record regarding work loss and productivity.

If, at the start of the trial, eligible subjects were on any prophylactic medication to treat their migraines, they entered a Washout Period of up to 14 days to taper from these medications. This washout was complete (that is, the prophylactic medications had lost all effect) by the time the subject entered the Prospective Baseline Period, 28 days prior to Visit 2. Eligible subjects who were not taking any prophylactic medications to treat their migraines did not enter the Washout Period, but immediately entered the Prospective Baseline Period.

At Baseline Visit 2 (Day 1), the headache/medication record information was reviewed. To be eligible for randomization into the trial a subject must have had 3 to 12 migraine periods but no greater than 15 (migraine and non-migraine) headache days during the 28 days prior to Visit 2. A headache day was defined as a calendar day during which the subject experienced headache pain of at least 30 minutes duration.

Double-Blind Phase:

Subjects who completed the Baseline Phase and met the entry criteria were randomized to one of four treatment groups: 50 mg/day topiramate, 100 mg/day topiramate, 200 mg/day topiramate or placebo. The Double-Blind Phase had two periods: Titration and Maintenance, which are described in more detail below.

Titration Period:

The Titration Period immediately followed the Baseline Phase and extended for eight weeks (56 days). During this period, subjects randomized to topiramate were started at a dose of 25 mg/day and the daily dose was increased by 25 mg weekly until they reach their assigned dose (or maximum tolerated dose, whichever is less). From the third week of Titration until the end of the Maintenance Period, a maximum of two dose level reductions were permitted for unacceptable tolerability problems. If the subject was still in the Titration Period after a dose reduction, rechallenge may have occurred in an attempt to achieve the subject's assigned dose, and, if unsuccessful, the dose may have been reduced again to the original reduced dose. Subjects who had their study medication dose decreased twice, and were still experiencing unacceptable tolerability problems that warranted additional dose reductions, exited the study. Clinic visits occurred on Day 29 (Visit 3) and Day 57 (Visit 4/End of Titration).

Maintenance Period:

During this 18-week period, the subjects remained on the dose of study medication reached at the end of the Titration Period (the assigned dose or the maximum tolerated dose). If the subject experienced unacceptable tolerability problems, the dose was reduced, only one dose reduction was permitted in the maintenance. No rechallenge was permitted during the Maintenance Period, so that subject continued on the reduced dose for the remainder of the period. Subjects who already had their study medication dose decreased by two levels, and were still experiencing unacceptable tolerability problems that warranted additional dose reductions exited the study. Clinic visits occurred on Day 85 (Visit 5), Day 113 (Visit 6), Day 141 (Visit 7) and Day 183 (Visit 8/Double-Blind Final Visit or Early Withdrawal).

Subjects were considered to have completed the Double-Blind Phase if they completed all 26 weeks of the Phase (8 weeks of Titration and 18 weeks of Maintenance) without prematurely discontinuing study medication.

Only subjects who had completed all 26 weeks of the Double-Blind Phase and/or exited the Double-Blind Phase for lack of efficacy (after completing at least 4 weeks of the Maintenance phase) were given the option of entering the Open-Label Extension Phase. Those subjects choosing not to enter the Open-Label Extension Phase were encouraged to complete the Taper/Exit Phase. Subjects who withdrew from the Double-Blind Phase for other reasons (lack of efficacy prior to completing 4 weeks of Maintenance, subject choice, adverse events) were not eligible to enter the Open-Label Extension Phase, but were encouraged to complete the Taper/Exit Phase.

Blinded Transition Phase:

Before eligible subjects entered the Open-Label Extension Phase, they first completed the Blinded Transition Phase.

During this phase, subjects were tapered from double-blind study medication in a blinded fashion while simultaneously titrating on open-label topiramate medication. The open-label titration rate was recommended as a weekly increase in the daily dose by 25 mg. This phase lasted for up to seven weeks depending on the dose achieved during the Double-Blind Phase. A clinic visit was scheduled the day after the taper from blinded medication was completed (Visit 10/End of Blinded Transition). Telephone follow-ups occurred periodically during the Transition Phase (e.g., every two weeks) to assess clinical outcome and/or adjust open-label dosing.

Open-Label Extension Phase:

This phase followed immediately the Blinded Transition Phase. Subjects received topiramate in an open-label fashion for up to six months, or until the subject withdrew. The open-label dose could be adjusted per the investigator's discretion, with the daily dose not exceeding 1600 mg. During this phase, multiple adjustments of the study medication were permitted to maximize efficacy or to minimize side effects. Subjects were seen quarterly during this phase (Visits 11 and 12/Open-Label Extension Final Visit). Telephone follow-ups occurred periodically to assess clinical outcome and/or adjust dosing.

Subjects were considered to have completed the Open-Label Extension Phase if they completed all six months of the Phase without prematurely discontinuing study medication.

Taper/Exit Phase:

It was recommended that all subjects exiting the studies taper from study medication. If subjects exited the studies during the Double-Blind Phase (Titration or Maintenance Period), they were tapered from study medication in a blinded fashion. The length of the taper varied according to the dose the subject achieved.

Subjects who exited the studies during the Blinded Transition Phase were tapered from open-label medication following the recommended taper schedule of 50-100 mg/week while simultaneously tapering from blinded medication.

Subjects who exited the studies during the Open-Label Extension Phase followed the recommended taper schedule of 50-100 mg/week.

A Follow-Up Visit (Visit 9 in the Double-Blind Phase and Visit 13 in the Open-Label Extension Phase) occurred within one week of all study medication being discontinued.

Dosage and Administration

Subjects were randomized to one of four treatment groups: a) placebo, b) 50 mg/day topiramate, c) 100 mg/day topiramate, or d) 200 mg/day topiramate. All subjects received study medication in a b.i.d. dosing regimen except during the first week of Titration, during which they took single evening doses.

Concominant Therapy

Ideally, no treatment other than study drugs and permitted medication designated by this protocol were used during the course of the studies. Because of the possible increased risk for renal stone formation from the following medications: acetazolamide, zonisamide and triamterene, it was recommended that they not be used in conjunction with topiramate therapy. It was also recommended that major tranquilizers (neuroleptics) tricyclic antidepressants, MAO inhibitors, or centrally acting sympathomimetics (e.g., dextroamphetamine sulfate [Dexedrine]) were not to be used in this trial.

Abortive/Rescue Medication

In keeping with good pain management practices, subjects enrolled in these studies were permitted to take acute abortive/rescue medication as indicated for the treatment of pain during migraine/headache attacks. The type and amount of medication used were recorded by the subject on the headache/medication record.

Allowable medications for the treatment of pain during migraine/headache attacks included the following, at the recommended dosage frequency:

No more than 15 treatment episodes per month: acetyl salicylic acid, acetaminophen, nonsteroidal anti-inflammatory agents, isometheptane mucate and acetaminophen, butalbital with aspirin and caffeine, butalbital with acetaminophen and caffeine.

No more than 8 treatment episodes/month: dihydroergotamine mesylate, ergotamine tartrate, codeine, codeine derivatives and triptans (either by injection, oral, or nasal spray).

No more than 6 treatment episodes/month: potent opioids such as meperidine/oxycodone.

No more than 2 treatment episodes/month corticosteroids for status migraine attacks.

A treatment episode was defined as a calendar day usage of a particular medication, (dosages allowable as per medication package insert)

If the use of rescue medications exceeded these frequencies, consideration was given to withdrawing the subject from the studies due to lack of efficacy and poor study compliance.

Medications taken for the relief of other migraine symptoms (e.g., vomiting, nausea) were permitted on a p.r.n. basis and were recorded on the headache record.

Study Evaluations

Physical examinations (including height) and neurologic examinations were performed at the beginning and end of the studies. A baseline electrocardiogram was performed at the beginning of the studies as well. Vital signs and weight were recorded at each clinic visit. Adverse events were recorded after study medication had been initiated and were followed until resolved or at a clinically stable endpoint. Clinical laboratory tests for all subjects, and urine pregnancy tests for females of childbearing potential were performed at selected intervals throughout the study. Quality of Life assessments were performed at Visits 2 (Day 1), 4 (Day 57/End of Titration), 6 (Day 113) and 8 (Day 183/Double-Blind Final Visit/Early Withdrawal). Health Care Resource Use information was recorded at Visits 3 through 8. The occurrence of any headaches or auras, severity and symptomatology of any headaches, and the use of abortive/rescue medication was transcribed from the subject's headache record to their case record form at each visit.

After Baseline Visit 1, subjects returned for scheduled visits within a window of +/−3 days until quarterly visits began (the Open-Label Extension Phase) at which time the window was +/−2 weeks.

Efficacy evaluations were based on information recorded on the subject's headache/medication record and Health-Related Quality of Life (HRQL) assessments. On the headache/medication record the subjects documented the following throughout his study participation: occurrence and duration of headaches (and auras if no headache pain develops), severity of headache pain and associated symptoms, as well as the use of medication taken to relieve headache pain or symptoms (or taken during an aura to relieve symptoms or prevent migraine pain). HRQL assessments were completed at specified intervals throughout the study (see Time and Events Schedule) by subjects 18 years or older at the time of study entry. Two instruments, the Migraine-Specific Quality of Life questionnaire (MSQ), and the Medical Outcomes Study Short Form-36 (SF-36) were used to assess HRQL.

The SF-36 is the most frequently used generic measure of HRQL in migraine patients and has been used in several studies of migraine. The SF-36 is a 36-item questionnaire measuring eight domains. The SF-36 has been shown to be reliable and valid in a wide variety of patient populations as well as for migraine patients.

The MSQ, developed by Glaxo Wellcome was also administered in these clinical trials. It is a disease-specific instrument developed to assess quality of life relating to migraine. The current version (2.1) has 14 items within three domains. The MSQ has been used most often in published clinical trials of migraine therapy and it has demonstrated evidence of reliability, validity, and responsiveness.

Efficacy Criteria

The primary efficacy endpoint was a change in monthly (28 days) migraine period rate from the Prospective Baseline Period to the Double-Blind Phase.

The secondary efficacy endpoints included the proportion of subjects responding to treatment (50% or more reduction in the monthly migraine period rate), the change in number of monthly migraine attacks (per IHS criteria) from the Prospective Baseline Period to the Double-Blind Phase, the change in monthly migraine days from the Prospective Baseline Period to the Double-Blind Phase, the change in number of days per month requiring rescue medication from the Prospective Baseline to the Double-Blind Phase, and HRQL assessments.

The efficacy criteria were primarily based on the superiority of one or more topiramate doses to placebo in terms of statistically significant difference in the primary endpoint. Secondary endpoints were used to support the conclusion based on the primary endpoint, and to evaluate the treatment effect on subjects' quality of life.

Efficacy Evaluations

The primary efficacy endpoint was the change in monthly (28 day) migraine period rate from the Prospective Baseline Period to the Double-Blind Phase. The secondary efficacy endpoints included: the proportion of subjects responding to treatment (50% or more reduction in the monthly migraine period rate from the Prospective Baseline Period to the Double-Blind Phase), the change in number of monthly migraine attacks (according to IHS criteria) from the Prospective Baseline Period to the Double-Blind Phase, the change in monthly migraine days from the Prospective Baseline Period to the Double-Blind Phase, and the change in number of days per month requiring rescue medication from the Prospective Baseline Period to the Double-Blind Phase. Other secondary efficacy variables included migraine-specific measures of health-related quality-of-life (MSQ) and SF-36 quality-of-life measures. All statistical tests were performed two-sided at a p value of less than or equal to 0.05 significance level unless otherwise specified.

Statistical analyses were primarily based on intent-to-treat principle. Intent-to-treat analysis population included all randomized subjects who reported data during the Double-Blind Phase. Missing data was imputed by using the value carrying forward (LVCF) approach. If the number of subjects with major protocol violations was not negligible, then a per-protocol analysis excluding subjects with major protocol violations was carried out to assess the robustness of the results. All protocol violations were identified, and a decision about the need for a per-protocol analysis was made prior to the unblinding of the database. The list of major protocol violations was included in a formal data analysis plan.

The primary efficacy endpoint, the change in monthly migraine period rate from the Prospective Baseline Period to the Double-Blind Phase, was assessed by a linear model with factors for baseline value, treatment, and study center. Comparisons of topiramate doses with placebo were made using the Tukey-Ciminera-Heyse trend test which is a step-down procedure including all doses and placebo at the first stage. If a significant trend in response with dose was detected, then the 200 mg dose was deemed significantly different from placebo and dropped from the trend test of the 100 mg dose, which included the 100 mg, 50 mg and placebo doses. If 100 mg dose was significantly different from placebo by the trend test, then 50 mg dose was compared with placebo. This trend test controls overall comparison type-I error in finding the minimal effective dose level. No further α-level adjustment was necessary, since there is only one primary endpoint. Secondary efficacy endpoints results were used to support the conclusion based on the primary efficacy endpoint and thus no multiplicity adjustments were applied. Treatment-by-center interactions were examined by graphical display of the results of individual centers and by the same linear model with an additional factor for treatment-by-center interaction at 0.10 significance level. Assumptions of normality and homogeneity were verified.

To address the dose-response relationship and to facilitate the discussion of dose selection, in addition to the above trend-test analysis, a secondary analysis to compare among topiramate doses was performed. Furthermore, confidence intervals and graphic methods were used to estimate the relationship between dose and the primary endpoint as well as the secondary endpoints.

The proportion of subjects responding to treatment was analyzed using the Cochran-Armitage trend test procedure. The change from baseline in number of monthly migraine attacks (per IHS criteria) and monthly migraine days and the change from baseline in number of days per month requiring rescue medication was assessed in the same way as that for the primary endpoint. No multiplicity adjustments were applied to the multiple secondary comparisons since the results from these secondary endpoints were used to support the conclusion based on the primary efficacy endpoint.

Data for all types of headache, migraine duration, severity of migraine headaches, and severity of migraine associated symptoms were summarized and/or analyzed if needed.

The primary HRQL analysis endpoints were the three MSQ domains: Role Restriction, Role Prevention, and Emotional Function. Secondary HRQL endpoints included the eight SF-36 domains: physical functioning, role-physical, bodily pain, general health, vitality, social functioning, role emotional and mental health as well as the SF-36 Physical Component Summary and the SF-36 Mental Component Summary.

Treatment group comparisons were performed for all HRQL scales. Multiple comparison probability adjustments were performed on the primary HRQL endpoints (the three MSQ domains) only, using a sequentially rejective Bonferonni adjustment procedure.

The HRQL hypotheses to be tested were: 1) prophylactic topiramate treatment associated with improved HRQL relative to placebo; and 2) improvements in HRQL associated with reductions in migraine frequency.

The primary analytic technique for testing between-group differences was based upon a longitudinal analysis of the HRQL through the end of double-blind treatment (Day 183). The longitudinal analysis utilized a piecewise linear regression model, allowing the slope of the HRQL curve to change at the end of titration (Day 57). Area under the curve analysis from randomization to Day 183 comprised the primary analysis comparing treatment groups. Sensitivity analyses were performed to test different assumptions relating to missing HRQL data (i.e., data missing at random or data missing not at random).

The association between change in migraine frequency and change in HRQL was examined using correlational techniques. Change in HRQL was defined as the absolute change in HRQL domain from baseline to last HRQL assessment. The change in migraine frequency was measured as the difference between the number of migraines during pre-randomization (Day-28 to Day 1) and the number of migraines in the last 28 day period the subject in the double-blind phase of the study. Multiple comparison adjustments were performed for the three primary HRQL endpoints only.

Sample Size Determination

A sample size of 120 per group gave 95% power to detect a treatment difference of 1.19 in change from baseline in migraine period rate between any pair of treatment groups assuming 2.50 as the common standard deviation. It was believed that 2.50 was a reasonable estimation of the high end of the variability of change from baseline in migraine period rate in the current study.

Results from the two clinical trials described above were analyzed for the effect of topiramate in combination with acute or rescue therapies, more specifically triptan rescue therapies, on the severity and duration of headaches as well as the severity of any associated symptoms of nausea, photophobia and/or phonophobia. Severity of headache and associated symptoms was evaluated by the clinical trial participants on a categorical scale of 0 to 3 (0=none, 1=mild, 2=moderate, 3=severe). Duration of headache was expressed in hours. The placebo controlled group was then compared with the groups treated with topiramate at 50 mg, 100 mg and 200 mg with results as listed in Tables 1-5 below. Statistical significance was calculated for the difference from placebo for the topiramate 100 mg treated group and for all topiramate treated groups combined. Statistical significant differences were those with a p value of less than or equal to 0.05.

TABLE 1
Severity of Migraine Headaches

| | | TPM | TPM | TPM | TPM |
|---|---|---|---|---|---|
| | Placebo | 50 mg | 100 mg | 200 mg | Total |
| Baseline | 2.27 | 2.32 | 2.33 | 2.26 | |
| Double-Blind | 2.23 | 2.18 | 2.14 | 2.16 | |
| Difference | −0.04 | −0.13 | −0.19 | −0.10 | |
| p value | | N/C | 0.007 | N/C | 0.082 |

N/C indicates that the p value was not calculated

TABLE 2
Duration of Migraine Headaches (in hours)

| | | TPM | TPM | TPM | Total |
|---|---|---|---|---|---|
| | Placebo | 50 mg | 100 mg | 200 mg | TPM |
| Baseline | 13.48 | 12.77 | 14.75 | 10.84 | |
| Double-Blind | 12.65 | 11.60 | 13.17 | 12.05 | |
| Difference | −0.82 | −1.17 | −1.59 | +1.21 | |
| p value | | N/C | 0.998 | N/C | 0.815 |

N/C indicates that the p value was not calculated

The results above indicate that topiramate in combination with triptan rescue medication resulted in a decrease in the severity of migraine headaches as compared to treatment with rescue medications alone. Numerical differences were measurable for patient groups taking 50 mg, 100 mg and 200 mg topiramate. Statistically significant results were measured for the patient group taking 100 mg topiramate.

The results further indicate that topiramate in combination with triptan rescue medications resulted in a numerically measurable decrease in the duration of a migraine headache in patient groups taking 50 mg and 100 mg topiramate. At this time the inventors do not have a reasonable theory as to why the duration of headaches increased at the 200 mg topiramate treatment. It is believed that if the studies had been designed to specifically evaluate the effect of a combination, this would not have been seem.

TABLE 3
Severity of Nausea Associated Migraine Headaches

| | | TPM | TPM | TPM | TPM |
|---|---|---|---|---|---|
| | Placebo | 50 mg | 100 mg | 200 mg | Total |
| Baseline | 1.16 | 1.17 | 1.22 | 1.12 | |
| Double-Blind | 1.25 | 1.17 | 1.12 | 1.09 | |
| Difference | +0.09 | −0.01 | −0.10 | −0.03 | |
| p value | | N/C | 0.045 | N/C | 0.201 |

N/C indicates that the p value was not calculated

The results above indicate that topiramate treatment resulted in a decrease in the severity of nausea associated with migraine headaches when acute or rescue medications of the triptan class were taken. The effect was numerically measurable (and greater than placebo) at 100 mg and 200 mg doses. Statistically significant differences in the severity of nausea were measured in patients taking 100 mg topiramate.

TABLE 4
Severity of Photophobia Associated Migraine Headaches

| | | TPM | TPM | TPM | TPM |
|---|---|---|---|---|---|
| | Placebo | 50 mg | 100 mg | 200 mg | Total |
| Baseline | 1.73 | 1.83 | 1.77 | 1.62 | |
| Double-Blind | 1.65 | 1.68 | 1.58 | 1.57 | |
| Difference | −0.08 | −0.14 | −0.19 | −0.05 | |
| p value | | N/C | 0.162* | N/C | 0.514 |

N/C indicates that the p value was not calculated
*Change from baseline in severity of photophobia for TPM at 100 mg achieved statistical significance for Study 1 (p = 0.011) but not for Study 2 (p = 0.626)

The results above indicate that topiramate treatment in combination with triptan rescue medication decreased the severity of associated photophobia (light sensitivity) relative to treatment with triptan rescue medications alone. The effect was numerically measurable (and greater than placebo) in the patient groups taking 50 mg and 100 mg topiramate. Statistically significant differences in the severity of nausea were measured in patients taking 100 mg topiramate in Study 1.

TABLE 5
Severity of Phonophobia Associated With Migraine Headaches

| | | TPM | TPM | TPM | TPM |
|---|---|---|---|---|---|
| | Placebo | 50 mg | 100 mg | 200 mg | Total |
| Baseline | 1.56 | 1.65 | 1.72 | 1.50 | |
| Double-Blind | 1.53 | 1.59 | 1.58 | 1.47 | |
| Difference | −0.03 | −0.06 | −0.15 | −0.03 | |
| p value | | N/C | 0.251* | N/C | 0.762 |

N/C indicates that the p value was not calculated
*Change from baseline in severity of phonophobia for TPM at 100 mg achieved statistical significance in Study 1 (p = 0.018) but not for Study 2 (p = 0.463).

The results above indicate that topiramate treatment in combination with triptan rescue medication decreased the severity of associated phonophobia (sound sensitivity) relative to treatment with triptan rescue medications alone. The effect was numerically measurable (and greater than placebo) in the patient groups taking 50 mg and 100 mg topiramate. Statistically significant differences in the severity of nausea were measured in patients taking 100 mg topiramate in Study 1.

Additional analyses of the results collected from the above study trials #1 and #2 are ongoing.

### Clinical Trial Protocol—Clinical Trial #3

### Randomized, Double-Blind, Placebo-Controlled, Parallel Group. Dose Response Study

The primary objective of the study was to evaluate the safety and efficacy of two doses of topiramate (100 and 200 mg/day) versus placebo in the prophylaxis of recurrent episodes of migraine based on change from the baseline phase to the double-blind phase in the monthly (28 days) migraine episode rate.

The secondary objectives were to a) evaluate the effect of prophylactic treatment with topiramate (100 and 200 mg/day) versus placebo in migraine patients on percentage of subjects responding to treatment (50% or more reduction in monthly migraine episode rate) and change from the baseline phase to the double-blind phase in b) migraine days per month, c) average migraine duration, d) rescue medication use, e) average severity of migraine headache, f) average severity of migraine associated symptoms (nausea, vomiting, photophobia, phonophobia); to provide safety and efficacy data for the comparison between topiramate (100 and 200 mg/day and propanolol (160 mg/day) in the prophylactic treatment of migraine; and to evaluate the effect of prophylactic treatment with topiramate (100 and 200 mg/day) versus placebo in migraine patients on migraine-specific measures of health-related quality of life (HRQL) and SF-36 quality-of-life measures, as well as the correlation between HRQL and migraine frequency.

This was a randomized, double-blind, placebo controlled, parallel-group, multicenter study to evaluate the efficacy and safety of two doses of topiramate versus placebo and propanolol in migraine prophylaxis. Five hundred seventy five male and female subjects were randomized to the four treatment groups. The subjects must have been diagnosed with migraine for at least twelve months, with or without aura, as defined by the International Headache Society (HIS).

The IHS diagnostic criteria differ from the definition of a migraine period utilized in this study for evaluation of efficacy. For the purposes of this study a migraine period was defined as the twenty-four hour duration starting with the onset of painful migraine symptoms, or aura with successful abortive/rescue treatment. Any recurrence during the twenty-four hour period was considered part of the initial episode. If the migraine pain persisted beyond the twenty-four hour period, for the purposes of this study, this was considered a new episode.

There were four phases in this study: Baseline, Core Double-Blind, Blinded Extension, and Taper/Exit, which are described in more detail below.

Baseline Phase:

The Baseline Phase lasts up to 42 days and included two periods: Washout and Prospective Baseline.

At Baseline Visit 1 (screening), subjects were evaluated to ensure that they met inclusion/exclusion criteria. In addition, a three month retrospective headache history was recorded. During each of the three months prior to Visit 1, subjects should have had no more than 8 migraines and no more than 15 total headache days (migraine plus other headache types). Eligible subjects then underwent other study procedures and were given a headache/rescue medication record. Subjects maintained this record from Visit 1 onward throughout their study participation, documenting the occurrence of any headaches, or auras, as well as the duration, severity and symptomatology of any migraine attacks. Subjects also recorded the use of any abortive/rescue medication taken for the relief of migraine pain and associated symptoms, or during an aura to prevent migraine pain or relieve symptoms. In addition, for each migraine attack, subjects answered the questions on the headache record regarding work loss and productivity.

If, at the start of the trial, eligible subjects were on any prophylactic medication to treat their migraines, they entered a Washout Period of up to 14 days to taper from these medications. This washout was completed (that is, the prophylactic medications had lost all effect) by the time the subject enters the Prospective Baseline Period, 28 days prior to Visit 2 (randomization).

Eligible subjects who were not taking any prophylactic medications to treat their migraines did not need to enter the Washout Period, but immediately entered the Prospective Baseline Period.

At Baseline Visit 2 (Day 1), the headache/rescue medication record information was reviewed. To be eligible for randomization into the trial a subject must have had 3 to 12 migraine episodes but no greater than 15 (migraine and non-migraine) headache days during the 28 days prior to Visit 2.

Core Double-Blind Phase:

Subjects who completed the Baseline Phase and met the entry criteria (including Prospective Baseline Period migraine/headache rate) were randomized to one of four treatment groups: 50 mg/day topiramate, 100 mg/day topiramate, 200 mg/day topiramate or placebo. The Core Double-Blind Phase had two periods: Titration and Maintenance, as follows:

Titration Period:

The Titration Period immediately followed the Baseline Phase and extended for eight weeks (56 days). During this period, subjects randomized to topiramate were started at a dose of 25 mg/day and the daily dose increased by 25 mg weekly until they reach their assigned dose (or maximum tolerated dose, whichever is less). Subjects randomized to propanolol were started at a dose of 20 mg/day and the daily dose increased by 20 mg weekly until they reach their assigned dose (or maximum tolerated dose, whichever was less). From the third week of Titration until the end of the Maintenance Period, a maximum of two dose level reductions were permitted for unacceptable tolerability problems. If the subject was still in the Titration Period, after a dose reduction, rechallenge was attempted to approach the subject's assigned dose, and, if unsuccessful, the dose was reduced again to the original reduced dose. Subjects who had already had their study medication dose decreased by two levels, and were still experiencing unacceptable tolerability problems which warranted additional dose reductions, exited the study, or entered the Open Label Extension Phase, where their dose was further adjusted. Clinic visits occurred on Day 29 (Visit 3) and Day 57 (Visit 4/End of Titration).

Maintenance Period:

During this 18-week period, the subjects remained on the dose of study medication reached at the end of the Titration Period (the assigned dose or the maximum tolerated dose). If the subject experienced unacceptable tolerability problems, the dose was reduced, but only to the point that there were no more than two dose reductions for the entire Core Phase (Titration plus Maintenance). No rechallenge was permitted during the Maintenance Period, so the subject continued on the reduced dose for the remainder of the period. Subjects who had already had their study medication dose decreased by two levels, and were still experiencing unacceptable tolerability problems which would warrant additional dose reductions, exited the study. Clinic visits occurred on Day 83 (Visit 5), Day 113 (Visit 6), Day 141 (Visit 7) and Day 183 (Visit 8/Core Double-Blind Final Visit or Early Withdrawal).

Subjects were considered as having completed the Core Double-Blind Phase if they complete all 26 weeks of the Phase (8 weeks of Titration and 18 weeks of Maintenance) without prematurely discontinuing study medication.

Only subjects who completed all 26 weeks of the Core Phase had the option of entering the Blinded Extension Phase.

Subjects who withdrew from the Core Phase for any reason, or who choose not to enter the Blinded Extension Phase completed the Taper/Exit Phase.

Blinded Extension Phase:

During this phase, subjects remained on their study medication at the same dose they achieved during the Core Phase for six months, or until they withdrew, or development was discontinued. During this phase, subjects were not permitted to adjust the dose of study medication. Subjects were seen quarterly during this phase (Visits 10 and 11/Blinded Extension Final Visit).

Subjects were considered as having completed the Blinded Extension Phase if they completed all six months of the Phase without prematurely discontinuing study medication.

Taper/Exit Phase:

Subjects exiting the study were tapered from study medication. If the subject exited the study during the Core Double-Blind Phase (Titration or Maintenance Period), they were tapered from study medication in a blinded fashion. The length of the taper was as long as seven weeks, but varied according to the dose the subject achieved.

Subjects who exited the study during the Blinded Extension Phase were tapered from their medication following the recommended taper schedule.

The investigator was permitted to accelerate the taper if clinically indicated for individual subjects. A follow-up visit (Visit 9 in the Core Phase and Visit 12 in the Blinded Extension Phase) occurred within one week after all study medication was discontinued.

Dosage and Administration

Subjects were randomized to one of four treatment groups: 100 mg/day topiramate, 200 mg/day topiramate, 160 mg/day propanolol, or placebo. All subjects received study medication on a b.i.d. dosing regimen except during the first week of Titration, during which they were taking a single evening dose.

Concominant Therapy

Ideally, no treatment other than study drugs and permitted medication designated by this protocol were to used during the course of the study. Because of the possible increased risk for renal stone formation from the following medication, it was recommended that the following medications: acetazolamide, zonisamide, amiloride and triamterene, were not used in conjunction with topiramate therapy: It was also recommended that major tranquilizers (neuroleptics) tricyclic antidepressants, MAO inhibitors, or centrally acting sympathomimetics (e.g., dextroamphetamine sulfate [Dexedrine]) were not to be used in this trial.

Rescue Medication

In keeping with good pain management practices, subjects enrolled in this study were permitted to take acute abortive/rescue medication as indicated for the treatment of migraine episodes. The type and amount of rescue medication used was recorded by the subject on the headache/rescue medication record.

Allowable medications for acute symptoms included the following at the recommended dosage frequency:

Antiemetics (p.r.n.)

No more than 15 days per month: acetyl salicylic acid (unless being given for cardiac vascular disease prophylaxis), acetaminophen, nonsteroidal anti-inflammatory agents, isometheptane mucate and acetaminophen, butalbital with aspirin and caffeine, butalbital with acetaminophen and caffeine

No more than 8 days/month: codeine, codeine derivatives and triptans (either by injection, oral, or nasal spray).

No more than 6 days/month: potent narcotics such as Demerol/Morphine

No more than 2 days/month: corticosteroids for status migraine attacks

No more than 8 days/month: Dihydroergotamine mesylate, Ergotamine Tartrabe (less than 10/week or 3/day)

If the use of rescue medications exceeded these frequencies, consideration was given to withdrawing the subject from the study due to lack of efficacy (if the subject has completed all eight weeks of titration, he has the option of entering the Open Label Extension Phase).

Excluded abortive/rescue medications included the following: other anticonvulsants, tricyclics, SSRI's (these may only be used at a stable dose for the treatment of diagnosed depression), major tranquilizers, transcutaneous stimulator, beta-blockers, illicit narcotics, propanolol, calcium channel blockers, Methysergide, Herbal preparations reputed to be useful in headache therapy (Examples: Fever Few, St. John's Wort), corticosteroids, local anesthetics, botulinum toxin injections used for the routine treatment of headache and riboflavin.

Study Evaluations

Physical examinations (including height) and neurologic examinations were performed at the beginning and end of the study. A baseline electrocardiogram was performed at the beginning of the study. Vital signs and weight were recorded at each clinic visit. Adverse events were recorded after study medication had been initiated and was followed until resolved or at a clinically stable endpoint. Clinical laboratory tests for all subjects, and urine pregnancy tests for females of childbearing potential were performed at selected intervals throughout the study. Quality of Life assessments wer performed at Visits 2 (Day 1), 4 (Day 57/Exit from Titration), 6 (Day 113) and 8 (Day 183/Core Double-Blind Final Visit/Early Withdrawal). Health Care Resource Use information was recorded at Visits 3 through 8. The occurrence of any headaches or auras, severity and symptomatology of any migraine headaches, and the use of rescue medication was transcribed from the subject's headache record to their case record form at each visit.

After Baseline Visit 1, subjects returned for scheduled visits within a window of +/−3 days until quarterly visits began (the blinded Extension Phase) at which time the window was +/−2 weeks.

Efficacy Evaluations

Efficacy evaluations were based on information recorded on the subject's headache/rescue medication record and Health-Related Quality of Life assessments. On the headache/rescue medication record the subjects documented the following throughout his/her study participation: occurrence and duration of headaches (and auras if no headache pain develops), severity of migraine pain and associated symptoms, as well as the use of medication taken to relieve migraine pain or symptoms (or taken during an aura to relieve symptoms or prevent migraine pain). Health-Related Quality of Life (HRQL) assessments were completed at specified intervals throughout the study (see Time and Events Schedule, page 9) by subjects 18 years or older at the time of study entry. Two instruments, the Migraine-Specific Quality of Life questionnaire (MSQ), and the Medical Outcomes Study Short Form-36 (SF-36) were used to assess HRQL.

Efficacy Criteria

The primary efficacy criteria was the reduction in migraine episodes per month (28 days) during the Core Double-Blind Phase compared to the 28 day Prospective Baseline Period.

The secondary efficacy criteria included the percentage of subjects responding to treatment (50% or more reduction in the monthly (28 day) migraine episode rate) and reduction from the Prospective Baseline Period to the Core Double-Blind Phase in a) migraine days per month, b) monthly rate of all types of headaches, c) average migraine duration, d) rescue medication use, e) average severity of migraine headache, and f) average severity of migraine-associated symptoms (nausea, vomiting, photophobia, phonophobia). Also included in the secondary efficacy criteria was the effect of prophylactic treatment with topiramate versus placebo on migraine-specific measures of health-related quality of life (HRQL) and SF-36 quality-of-life measures, as well as the correlation between HRQL and migraine frequency.

The study also provided safety and efficacy data for the comparison between topiramate (100 and 200 mg/day) and propanolol (160 mg/day) in the prophylactic treatment of migraine.

The Medical Outcomes Study Short Form-36 (SF-36) is the most frequently used generic measure of HRQL in migraine patients and has been used in several studies of migraine. The SF-36 is a 36-item questionnaire measuring eight domains. The SF-36 has been shown to be reliable and valid in a wide variety of patient populations as well as for migraine patients.

The migraine specific quality of life questionnaire (MSQ), developed by Glaxo Wellcome was also administered in this clinical trial. The MSQ is a disease-specific instrument developed to assess quality of life relating to migraine. The current version (2.1) has 14 items within three domains. The MSQ has been used most often in published clinical trials of migraine therapy and it has demonstrated evidence of reliability, validity, and responsiveness.

Completion

Subjects were considered as having completed the Core Double-Blind Phase if they completed the entire 26 weeks of the Phase (8 weeks of Titration plus 18 weeks of Maintenance) without prematurely discontinuing study medication. Subjects who withdrew from the study for any reason before completion of this phase were not considered to have completed.

Subjects were considered as having completed the Blinded Extension Phase if they completed the entire six months of the phase without prematurely discontinuing study medication.

Subject participation may have been terminated prior to completing the Core Double-Blind Phase for any of the following reasons: Adverse Event, Subject choice, Lost to follow-up, Lack of efficacy, or Other. When a subject withdrew prior to completing the study, the reason for withdrawal was documented on the CRFs and in the source document.

Efficacy Evaluations

The primary efficacy endpoint was the change in monthly (28 days) migraine episode rate from the Prospective Baseline Period to the Core Double-Blind Phase. The Double-Blind phase included both the Titration and Maintenance Periods.

The secondary efficacy endpoints included the percentage of subjects responding to treatment (defined as a 50% or more reduction in monthly migraine episode rate from the Prospective Baseline Period to the Core Double-Blind Phase); the change in migraine days per month (28 days) from the Prospective Baseline Period to the Core Double-Blind Phase; the change in the monthly (28 days) rate of all types of headache from Prospective Baseline Period to the Core Double-Blind Phase; the change in average migraine duration per episode from Prospective Baseline Period to the Core Double-Blind Phase; the change in number of days needing rescue medication per month (28 days) from the Prospective Baseline Period to the Core Double-Blind Phase; the change in average severity of migraine headache from Prospective Baseline Period to the Core Double-Blind Phase; and the change in average severity of migraine associated symptoms (nausea, vomiting, photophobia, phonophobia) from the baseline phase to the double-blinded phase. Other secondary efficacy variables included migraine-specific measures of health-related quality-of-life (MSQOL) and SF-36 quality-of-life measures.

Assessment of Efficacy:

Efficacy of topiramate in the prophylaxis of recurrent episodes of migraine was primarily demonstrated by a showing that a topiramate dose groups (100 mg and/or 200 mg/day) was superior to the placebo group based on change from the baseline phase to the double-blind phase in the monthly (28 days) migraine episode rate. In addition, the propranolol treatment group was included to provide data for the assessment of relative efficacy of topiramate vs propranolol treatment.

Analyses for Assessing Efficacy of Topiramate 100 mg and 200 mg vs. Placebo:

Statistical analyses were primarily based on intent-to-treat principle. These intent-to-treat analyses included all randomized subjects who reported data at least one time and took medication during the Double-Blind Phase. Missing data was imputed by using last value carrying forward (LVCF) approach.

The primary efficacy endpoint, the change in monthly migraine episode rate from the Prospective Baseline Period to the Core Double-Blind Phase, was assessed by a linear model with factors for baseline value, treatment, study site, and treatment-by-site interaction. Comparisons of topiramate doses with placebo was made using the Tukey-Ciminera-Heyse trend test which is a step-down procedure including all topiramate doses and placebo at the first stage. If a significant trend in response with dose is detected, then the 200 mg dose was deemed significantly different from placebo and dropped from the trend test of the 100 mg dose, which included the 100 mg and placebo doses. This trend test controls overall comparison type-I error in finding the minimal effective dose level for each efficacy endpoint. Secondary efficacy endpoints results were used to confirm and support the conclusion based on the primary efficacy endpoint. Treatment-by-site interaction was examined at 0.010 significance level.

All secondary endpoints (except for the percentage of responders) were assessed in the same way as that for the primary endpoint. The percentage of subjects responding to treatment was analyzed using the Cochran-Armitage trend test procedure. Consistence of topiramate dose-related treatment effects across different subgroups (gender, age, etc.) was explored.

Comparison between Propranolol and Placebo to Establish Assay Sensitivity:

The propranolol group was compared with placebo group based on the primary endpoint data to validate current trial's assay sensitivity.

Assessing Efficacy of Topiramate 100 mg and 200 mg Relative to that of Propranolol 160 mg/day:

Summary statistics of the change of monthly migraine episode rate and 95% confidence interval for the difference between topiramate (100 mg and 200 mg) group and propranolol group were provided for the assessment of the similarity in efficacy.

Sample Size Determination

Sample size of 120 per group gave 95% power to detect a treatment difference of 1.19 in change from baseline in migraine episode rate between any pair of treatment groups assuming 2.50 as the common standard deviation. The use of 2.50 was an estimation of the standard deviation of change from baseline in migraine episode rate.

Thus, for treating and/or preventing migraine and/or any associated nausea, vomiting, photophobia, phonophobia or other symptoms, one or more compounds of formula (I) may be administered as co-therapy with one or more anti-migraine agents. Preferably, the co-therapy comprises administration of a therapeutically effective amount of a compound selected from the group consisting of antidepressants, beta blockers and triptans with topiramate. More preferably, the co-therapy comprises administration of a therapeutically effective amount of topiramate and a triptan.

Wherein the compound of formula (I) is topiramate, the topiramate is preferably administered in an amount in the range of about 10 to about 650 mg daily, more preferably in an amount in the range of about 25 to about 325 mg once or twice daily. Topiramate is currently available in unit dosage forms of 15 mg, 25 mg, 100 mg and 200 mg.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method for treating migraine comprising co-therapy, to a subject in need thereof, with a therapeutically effective amount of a triptan and a compound of the formula (I): 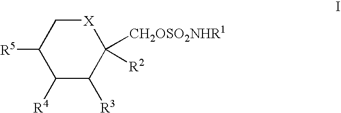
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (I)I: 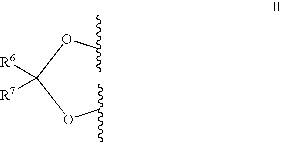
wherein
R⁶ and R⁷ are the same or different and are hydrogen, lower alkyl or are alkyl and are joined to form a cyclopentyl or cyclohexyl ring.

2. The method of claim 1 wherein the compound of formula (I) is topiramate.

3. The method of claim 2, wherein the amount of topiramate is from about 10 to about 650 mg daily.

4. The method of claim 3, wherein the amount of topiramate is from about 25 to about 325 mg once or twice daily.

5-12. (canceled)

13. The method of claim 1 wherein the triptan is selected from the group consisting of sumatriptan, naratriptan, rizatriptan, zolmitriptan, eletriptan, frovatriptan and almotriptan.

14. A method for treating the nausea, photophobia or phonophobia associated with a migraine headache comprising co-therapy, to a subject in need thereof, with a therapeutically effective amount of a triptan and a compound of the formula (I): 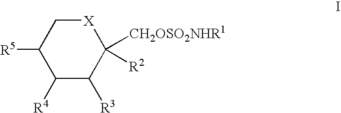
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (I)I: 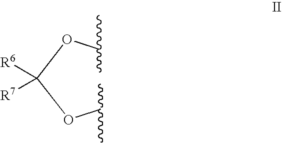
wherein
R⁶ and R⁷ are the same or different and are hydrogen, lower alkyl or are alkyl and are joined to form a cyclopentyl or cyclohexyl ring.

15. The method of claim 14 wherein the compound of formula (I) is topiramate.

16. The method of claim 15, wherein the amount of topiramate is from about 10 to about 650 mg daily.

17. The method of claim 16, wherein the amount of topiramate is from about 25 to about 325 mg once or twice daily.

18-20. (canceled)

21. The method of claim 14 wherein the triptan is selected from the group consisting of sumatriptan, naratriptan, rizatriptan, zolmitriptan, eletriptan, frovatriptan and almotriptan.

22. A method for preventing migraine comprising co-therapy, to a subject in need thereof, with a therapeutically effective amount of a triptan and a compound of the formula (I): 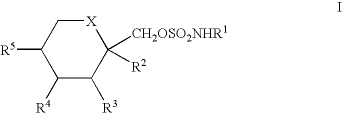
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula (I)I: 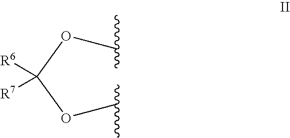
wherein
R⁶ and R⁷ are the same or different and are hydrogen, lower alkyl or are alkyl and are joined to form a cyclopentyl or cyclohexyl ring.

23. The method of claim 22 wherein the compound of formula (I) is topiramate.

24. The method of claim 23, wherein the amount of topiramate is from about 10 to about 650 mg daily.

25. The method of claim 24, wherein the amount of topiramate is from about 25 to about 325 mg once or twice daily.

26-30. (canceled)

31. The method of claim 22, wherein the triptan is selected from the group consisting of sumatriptan, naratriptan, rizatriptan, zolmitriptan, eletriptan, frovatriptan and almotriptan.

32. The method of claim 31, wherein the triptan is almotriptan.

33. The method of claim 13, wherein the triptan is almotriptan.

34. The method of claim 21, wherein the triptan is almotriptan.

35. The method of claim 2, wherein the triptan is selected from the group consisting of sumatriptan, naratriptan, rizatriptan, zolmitriptan, eletriptan, frovatriptan and almotriptan.

36. The method of claim 35, wherein the triptan is almotriptan.

37. The method of claim 15, wherein the triptan is selected from the group consisting of sumatriptan, naratriptan, rizatriptan, zolmitriptan, eletriptan, frovatriptan and almotriptan.

38. The method of claim 37, wherein the triptan is almotriptan.

39. The method of claim 23, wherein the triptan is selected from the group consisting of sumatriptan, naratriptan, rizatriptan, zolmitriptan, eletriptan, frovatriptan and almotriptan.

40. The method of claim 39, wherein the triptan is almotriptan.
